(19) United States
(12) Patent Application Publication
(10) Pub. No.: US 2011/0235889 A1
(43) Pub. Date: Sep. 29, 2011

(54) METHOD FOR ENHANCED VISUALIZATION OF OBJECTS IN INTERVENTIONAL ANGIOGRAPHIC EXAMINATIONS AND DEVICE FOR PERFORMING THE METHOD

(76) Inventor: Martin Spahn, Erlangen (DE)

(21) Appl. No.: 13/070,567

(22) Filed: Mar. 24, 2011

(30) Foreign Application Priority Data
Mar. 29, 2010 (DE) 10 2010 013 221.7

(51) Int. Cl. G06K 9/00; A61B 6/00; A61B 6/02

(52) U.S. Cl. 382/132; 378/62; 378/42

(57) ABSTRACT

A method for enhanced visualization of objects in interventional angiographic examinations is proposed. An empty image, a fill image with contrast-agent-filled vascular tree, and a native image and/or one further image with introduced object are acquired by a detector having a matrix-shaped array of pixels. The empty image is subtracted from the fill image to generate a subtraction image. The subtraction image is displaced by at least one pixel in the x- and/or y-direction and subsequently summed to generate a modified vessel image as a mask which has a substantially improved signal-to-noise ratio. The vascular tree in the modified vessel image is segmented to generate a segmentation image. The modified vessel image, the segmentation image with vascular tree, and the native image and/or further image are processed to generate a composite image. The composite image is played back.

### CROSS REFERENCE TO RELATED APPLICATIONS

This application claims priority of German application No. 10 2010 013 221.7 filed Mar. 29, 2010, which is incorporated by reference herein in its entirety.

### FIELD OF THE INVENTION

The invention relates to a method for enhanced visualization of objects in interventional angiographic examination and an angiographic X-ray system for performing the method.

### BACKGROUND OF THE INVENTION

For diagnostic examination purposes and for interventional procedures, for example in cardiology, radiology and neurosurgery, interventional X-ray systems are used for imaging, the typical major features of which can be a C-arm on which are mounted an X-ray tube and an X-ray detector, a patient positioning table, a high-voltage generator for generating the tube voltage, a system control unit, and an imaging system including at least one monitor. The C-arm can be held by means of a robotic arm, for example. A C-arm X-ray system of said type, as shown by way of example in FIG. 1, has a C-arm 2 which is rotatably mounted on a stand in the form of a six-axis industrial or articulated-arm robot 1 and at the ends of which are mounted an X-ray radiation source, for example an X-ray tube assembly 3 with X-ray tube and collimator, and an X-ray image detector 4 as the image recording unit.

The articulated-arm robot 1 known from U.S. Pat. No. 7,500,784 B2, for example, which preferably has six axes of rotation and hence six degrees of freedom, enables the C-arm 2 to be moved arbitrarily in space, for example by being rotated around a center of rotation between the X-ray tube assembly 3 and the X-ray detector 4. The inventive X-ray system 1 to 4 can be rotated in particular around centers of rotation and axes of rotation in the C-arm plane of the X-ray image detector 4, preferably around the center point of the X-ray image detector 4 and around axes of rotation intersecting the center point of the X-ray image detector 4.

The known articulated-arm robot 1 has a base frame which is permanently installed on a floor for example. Attached thereto is a turntable which is rotatable about a first axis of rotation. Mounted on the turntable so as to be pivotable about a second axis of rotation is a robotic floating link to which is attached a robotic arm which is rotatable about a third axis of rotation. Mounted at the end of the robotic arm is a robotic hand which is rotatable about a fourth axis of rotation. The robotic hand has a retaining element for the C-arm 2, said retaining element being pivotable about a fifth axis of rotation and rotatable about a sixth axis of rotation running perpendicular thereto.

The X-ray diagnostic apparatus is not dependent for its implementation on the industrial robot. Conventional C-arm devices can also be used. It is also possible to use biplane systems consisting, for example, of two C-arm X-ray systems as shown in FIG. 1.

The X-ray image detector 4 can be a flat semiconductor detector, rectangular or square in shape, which is preferably produced from amorphous silicon (a-Si). Integrating and possibly counting CMOS detectors can also be used, however.

A patient 6 to be examined is placed as the examination subject on a patient positioning table 5 in the beam path of the X-ray tube assembly 3 so that images of the patient's heart, for example, can be recorded. Connected to the X-ray diagnostic apparatus is a system control unit 7 having an imaging system 8 which receives and processes the image signals from the X-ray image detector 4—control elements are not shown, for example. The X-ray images can then be viewed on a monitor 9.

Important methods in imaging using C-arm X-ray systems are
diagnostic imaging with
   cardangiography at medium X-ray doses, a native visualization of the coronary vessels with the aid of contrast agents,
   digital subtraction angiography (DSA) for visualizing static vessels or vessels exhibiting little movement with the aid of contrast agents, wherein a native image is subtracted as a so-called “mask” from a series of native images in which a vessel or vascular tree is filled with contrast agent, the anatomical background disappearing as a result of the subtraction and the vessel or vascular tree alone remaining visible, and
   3D imaging with or without contrast agent, and
interventional imaging with
   fluoroscopy or real-time X-ray imaging, wherein primarily the positioning of catheters, guide wires, balloon catheters, stents, etc. is carried out at a low X-ray dose, this method also being employed purely diagnostically in order to position a catheter for administration of contrast agent, and
   roadmapping, wherein, similarly to DSA, a mask, a native image with contrast-agent-filled vascular tree, is produced initially. A series of native images is then generated in which a wire, for example, is moved. All anatomical structures disappear in order to subtract the mask image. Only the vascular tree and the wire moving “therein” remain visible.

An X-ray diagnostic apparatus for generating e.g. real-time X-ray images is described in US 2007/0201609 A1. The imaging unit generates 3D images of the blood vessels and of the remaining parts of the body which are not blood vessels. A projection processing circuit generates projection image data from the image portions of the remainder of the body and generates a blood vessel projection image from the blood vessel image. A device for identifying a position displacement determines a displacement between the X-ray images and the projection images of the parts of the body that are not blood vessels. A display plays back the X-ray images and the projection image of the blood vessels.

DE 101 22 875 C2 relates to a 3D angio-volume reconstruction method for a three-dimensional object based on recorded 2D projection images while avoiding shadow artifacts, wherein a 3D angio-volume dataset based on 2D mask images and a 3D angio-volume dataset based on 2D fill images are produced and the vascular tree is isolated in the fill volume dataset by means of a segmentation and after scaling is added to the mask image volume dataset.

US 2009/0192385 A1 discloses an X-ray diagnostic apparatus having a C-arm for generating virtual roadmap images in which CT-like image data of a patient is acquired with and without contrast agent and subtracted from one another in order to obtain a roadmap mask. The roadmap mask is acquired in the form of a 2D digitally reconstructed radiograph (DRR) from the same orientation as that of a fluoroscopy X-ray diagnostic apparatus which acquires real-time images of a patient during the latter's treatment. The fluoroscopic images are subtracted from the roadmap mask in order to enable the position of a catheter introduced into the body of the patient to be visualized more distinctly. If the orientation of the fluoroscopic images changes, the roadmap images of the corresponding orientation are used.

US 2008/0212857 A1 relates to a method for post-processing a 3D image dataset of a vascular structure of a body, wherein a 2D DSA of the vessel structure is recorded and registered with the 3D image dataset. The 2D DSA is compared with a corresponding projection image computed from the 3D dataset and the latter is modified, for example by changing the segmentation parameters, in order to adapt it to the 2D DSA.

US 2007/005878 A1 describes an X-ray diagnostic apparatus for generating image data of an examination subject, which apparatus has an image processing circuit that generates a plurality of reference image datasets and fluoroscopic image datasets. The reference images are acquired after injection of a contrast agent into the examination subject. Subsequently fluoroscopic roadmap images are generated from the reference images and the fluoroscopic images whose image directions correspond to one another.

There are further methods, such as 3D roadmapping for example, in addition to the methods cited here.

In a known roadmap method, as shown for example in FIG. 2, the following images are generated: a pure native image 10 (anatomy only) during the system dose regulation phase, a mask image 11, a native image from the fill phase, in which the vascular tree 12 is filled with contrast agent, and an image series 13 of native images, in which an object 14, for example a wire, is moved in the vascular tree 12 under fluoroscopy. The mask image 11 with contrast-agent-filled vascular tree 12 is subtracted in a subtraction stage 15 from the fluoroscopic image series 13 in which the object 14 can be seen and if necessary a constant K for setting the mean grayscale value is added in an addition stage 16. Further image processing steps such as contrast adjustment, edge enhancement, etc. can follow until an up-to-date subtraction series 17 is obtained in which only the moving object 14 is still readily identifiable in the “frozen” vascular tree 12.

In this case the method according to the invention is based, not on the normal, simple roadmap method described with reference to FIG. 2, but in principle on an extended roadmap method which is based on double subtraction, i.e. a subtraction both to generate the mask and to generate the “interventional” image series. This method is described below.

FIG. 3 shows a roadmap method using double subtraction, wherein the pure native image 10 is generated during the system dose regulation phase, the mask image 11 is generated from at least one native image from the fill phase, in which the vascular tree 12 is filled with contrast agent, and an image series 13 of native images, in which an object 14, for example a wire, is moved in the vascular tree 12 is generated under fluoroscopy, as described in the non-prior-published German patent application 10 2009 037 243.1.

The native image 10 and the mask image 11 are now subtracted one from the other in a first subtraction stage 20. A constant K for setting the mean grayscale value is added in a first addition stage 21 so that a first subtraction image 22 is obtained in which only the vascular tree 12 is to be seen. In a following first image processing stage 23 the visibility of the vascular tree 12 is improved so as to obtain an optimal vessel image 24.

In parallel therewith, the native image 10 and at least one of the native images of the image series 13 are subtracted from one another in a second subtraction stage 25. A constant K for setting the mean grayscale value, which can be different from the first constant K, is added in turn in a second addition stage 26. This yields a second subtraction image 27 in which only the object 14 can be seen. In a following second image processing stage 28 the visibility of the object 14 is improved such that at least one optimal object image 29 is obtained.

The vessel image 24 and the object image 29 are subtracted in a third subtraction stage 30 and a constant K for setting the mean grayscale value, which can be different from the other constants K, is added to the result in a third addition stage 31 such that optimal roadmap images 33 are obtained as a subtraction series after a third image processing stage 32 where necessary.

### SUMMARY OF THE INVENTION

The object of the invention is to embody a method in such a way that
during roadmapping the traditional drawbacks such as poor visibility of the wire, “burnout” (disappearance of the wire, which is rendered as dark, in the vascular tree, which is rendered as light, due to excessively high contrast), etc. are reduced or avoided, and
when a fluoroscopic image is overlaid over a DSA image (overlay reference), whereby the inverted DSA image is blended at a selectable percentage with a fluoroscopic image, the DSA image serving to visualize the vascular tree, the fluoroscopic image serving to visualize the wire being moved in the vascular tree, the visualization is improved.

The object is achieved according to the invention for a method and for a device by the features recited in independent claims. Advantageous embodiments are disclosed in the dependent claims.

The method proposes that firstly, in a first phase, X-ray images with pure anatomy are recorded during the system dose regulation phase and then, during a second phase, the fill phase, in which the vessels are filled with contrast agent, X-ray images are recorded, the mask image being produced from both sets of images. In a third phase, a working or intervention phase, X-ray images are produced under fluoroscopy while an object, a wire, a catheter or a “coil” for example, is moved in the vessel. Roadmap or overlay reference images are produced by subtraction and where appropriate further image processing techniques.

The angiographic X-ray system for performing the method has a C-arm X-ray system, on the C-arm of which are disposed an X-ray tube assembly and an X-ray detector having a matrix-shaped array of pixels for capturing at least one empty image, at least one fill image with contrast-agent-filled vascular tree, and at least one native image with introduced object, having an imaging system for receiving the output signals of the X-ray detector, having storage means, and having a monitor for playing back the signals processed by the imaging system.

In the case of an aforesaid method this is achieved by means of the following steps:
a) acquiring at least one empty image (10), at least one fill image (11) with contrast-agent-filled vascular tree (12), and at least one native image (13) and/or a further image with introduced object (14) by means of a detector (4) having a matrix-shaped array of pixels,
b) subtracting (20) the empty (10) and fill image (11) in order to generate a subtraction image (22),
c) displacing (35) the subtraction image (22) by at least one pixel in the x- and/or y-direction and subsequent summation in order to generate a modified vessel image (39) as a mask having a substantially improved signal-to-noise ratio,
d) segmenting (40) the vascular tree (12) in the modified vessel image (39) in order to generate a segmentation image (41),
e) processing (42) the modified vessel image (39), the segmentation image (41) with vascular tree (12), and at least one native image (13) and/or further image (29, 13) in order to generate at least one composite image (43), and
f) playing back the at least one composite image (33).

For a roadmap method it has proved advantageous if steps b) and e) are modified as follows:
b₁) subtracting (20) the empty (10) and fill image (11) in order to generate a first subtraction image (22),
b₂) subtracting (25) the empty image (10) and the at least one native image (13) in order to generate at least one second subtraction image (27),
b₃) processing (28) the at least one second subtraction image (27) in order to form at least one object image (29), and
e) processing (42) the modified vessel image (39), the segmentation image (41) with vascular tree (12), and the at least one object image (29) in order to generate at least one roadmap image as a composite image (43).

During the processing for generating at least one composite image the modified vessel image as the new mask image M′ and at least one further image as the fluoroscopic image series Fₙ can advantageously be overlaid with the aid of the binary information B of the segmentation image in a precisely targeted manner by merging in an overlay reference method.

According to the invention the processing of the modified vessel image and the at least one further image for the purpose of generating at least one composite image can be performed only in the region of the vascular tree in the segmentation image. This avoids the noise level being increased unnecessarily in the remaining regions. Said regions in fact contain no information, are therefore “zero” except for noise, since here anatomy has been subtracted from anatomy.

It has proved advantageous if, at the beginning of the processing in the at least one further image, the contrast is increased only in the region of the vascular tree in the segmentation image. The anatomy-corrected fluoroscopic image, the subtraction image Sₙ, is increased in contrast prior to the subtraction of the mask, and moreover in turn only at the point B(x,y)=1. This increase in contrast can be performed by means of a linear or non-linear function. Advantage: The visibility of the wire in the final image Rₙ or composite image is improved. By restricting the increase in contrast to the segmented region the noise in the remainder of the image is not made worse unnecessarily.

The segmentation image can advantageously selectively control filter methods in the image processing stages.

In this case spatial filter methods such as noise suppression techniques and/or sharpness filters can be selected as filter methods by means of the segmentation image in the image processing stages, i.e. the choice of parameters adapted as a function of the binary value.

According to the invention the filter methods can also be selected by means of the segmentation image in the image processing stages to treat the vascular and non-vascular regions separately.

It has proved advantageous if the filter methods are selected by means of the segmentation image in the image processing stages differently for the modified vessel image or mask image M′ and the interventional images Sₙ or Fₙ and/or the at least one further image.

The object is achieved according to the invention in the case of a device for performing the above-cited method by means of an angiographic X-ray system having a C-arm X-ray system, on the C-arm of which are mounted an X-ray tube assembly and an X-ray detector having a matrix-shaped array of pixels for acquiring at least one empty image, at least one fill image with contrast-agent-filled vascular tree and at least one native image and/or a further image with introduced object, having an imaging system for receiving the output signals of the X-ray detector, having storage means, and having a monitor for playing back the signals processed by the imaging system. According to the invention the imaging system additionally has a subtraction stage for subtracting the empty image and a mask image such that a subtraction image is obtained in which only the vascular tree can be seen, a displacement stage for displacing the subtraction image by at least one pixel in the x- and/or y-direction, an addition stage for subsequent summation of the displaced subtraction images for a modified vessel image as a mask which has a substantially improved signal-to-noise ratio, a segmentation stage of the modified vessel image for generating a segmentation image with extracted vascular tree and a further image processing stage for the modified vessel image, the segmentation image with vascular tree, and at least one further image for generating at least one composite image.

It has proved advantageous if the imaging system has a further subtraction stage for subtracting the at least one empty image and the at least one native image in order to generate at least one second subtraction image, a second image processing stage for processing the at least one second subtraction image in order to form at least one object image and the further image processing stage (42) for processing the modified vessel image (39), the segmentation image (41) with vascular tree (12) and the at least one native image (13) and/or object image (29) in order to generate at least one roadmap image as a composite image (43).

Alternatively, in order to generate at least one composite image, the further image processing stage can overlay the modified vessel image as a new mask image M′ and at least one further image as fluoroscopic image series Fₙ with the aid of the binary information B of the segmentation image in a precisely targeted manner by merging.

### BRIEF DESCRIPTION OF THE DRAWINGS

The invention is explained in more detail below with reference to exemplary embodiments shown in the drawing, in which:

FIG. 1 shows a known X-ray C-arm system for radiology, cardiology or neurosurgery having an industrial robot as carrier device,

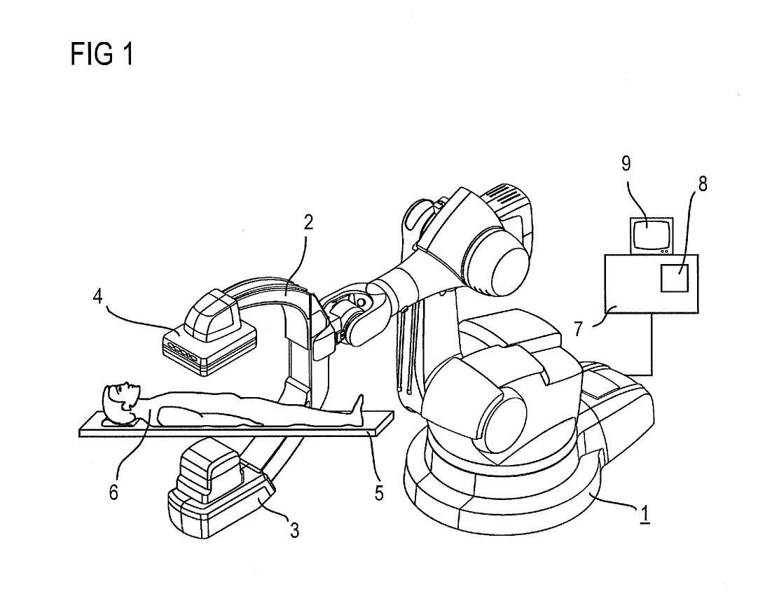

FIG. 2 shows a known roadmap method (state-of-the-art) with phase A “mask generation” and phase B “working phase”,

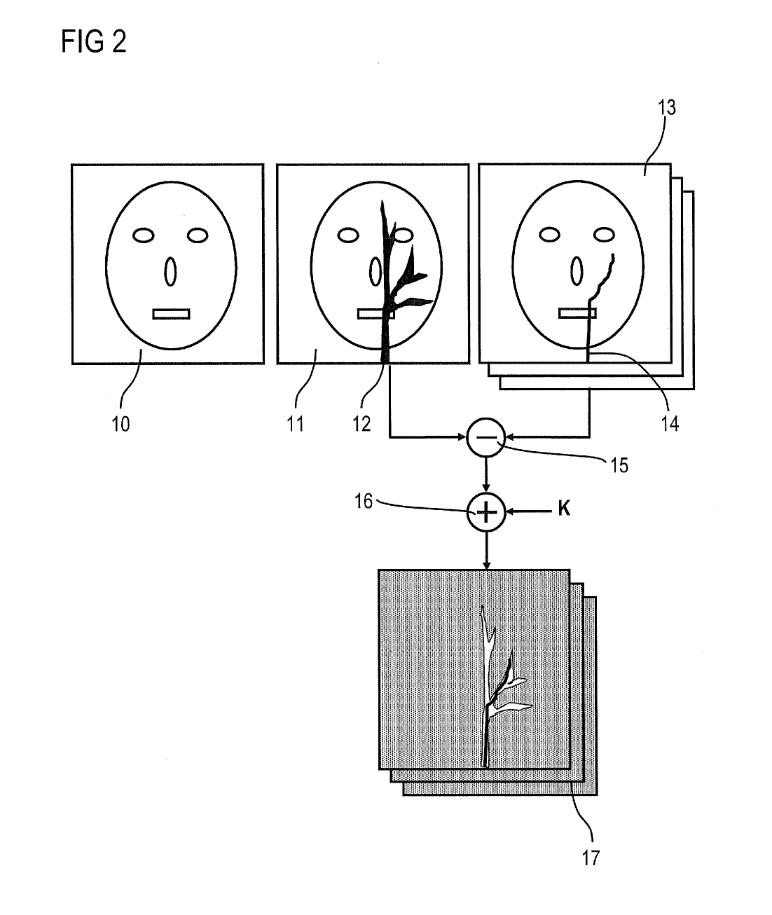

FIG. 3 shows a known roadmap method (state-of-the-art) with double subtraction imaging,

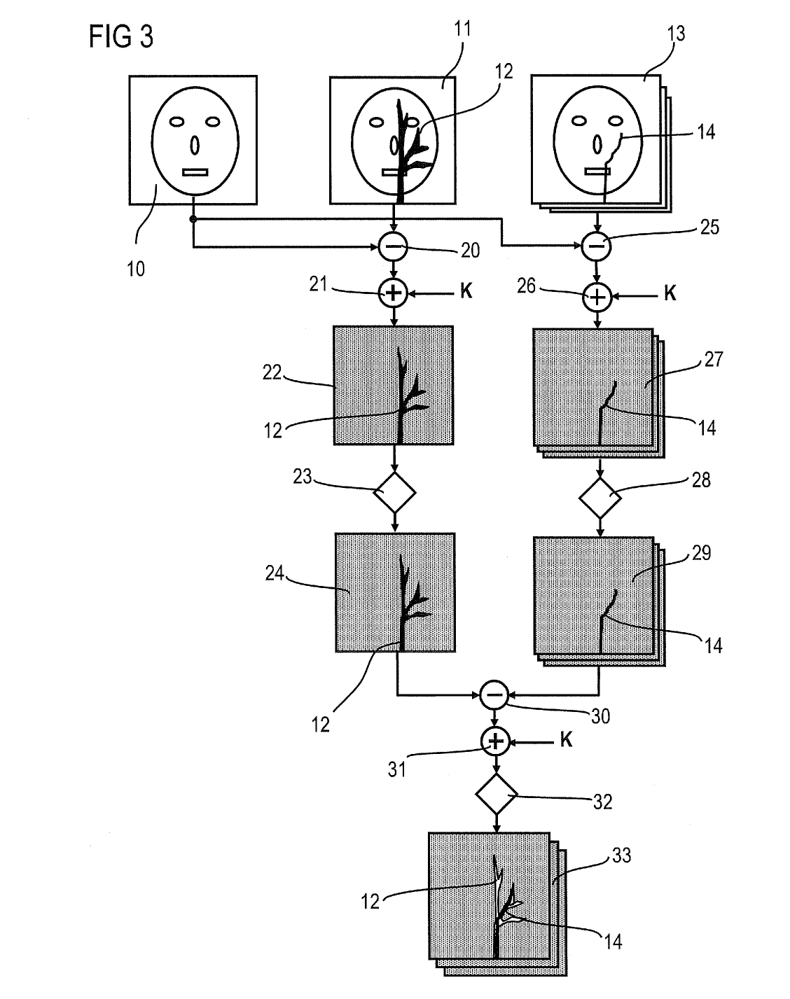

FIG. 4 shows a first embodiment variant of a roadmap method according to the invention, wherein by means of a self-improving signal-to-noise ratio the mask offers an improved means of segmenting the vascular tree which improves the further image processing,

FIG. 5 shows a second embodiment variant of a roadmap method according to the invention based on a new overlay reference method in which a segmentation image is produced from the mask and can be used as input for the further image processing and image overlaying, and

FIG. 6 shows a segmentation image B with “soft” transition between vascular and remaining region.

### DETAILED DESCRIPTION OF THE INVENTION

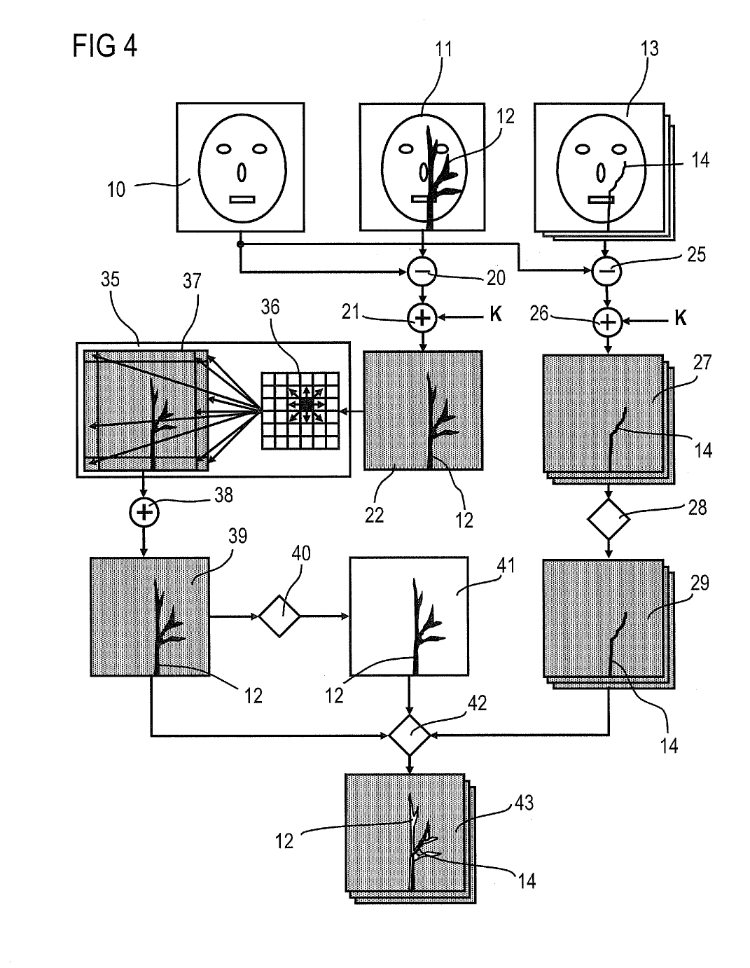

FIG. 4 shows a first embodiment variant of the method according to the invention, wherein a roadmap method using double subtraction is employed and wherein, by displacement of the original mask by one or more pixels in the x- and/or y-direction and subsequent summation, a mask is produced which has a substantially improved signal-to-noise ratio and from which the vascular tree can be segmented.

Instead of the first image processing stage 23 of the first subtraction image 22 in which only the vascular tree 12 can be identified, by means of which the visibility of the vascular tree 12 is improved, the first subtraction image 22 is supplied as mask M to a displacement stage 35 which, as indicated by the displacement symbol 36, effects a displacement of the subtraction image 22 by at least one pixel in the x- and/or y-direction, such that a plurality of vessel images 37 displaced relative to one another is obtained which are summed by means of a further addition stage 38 in order to generate a modified vessel image 39 as a new mask M′ and in the process are correctly normalized.

This modified vessel image 39 is converted by means of a segmentation stage 40 into a binary image B or segmentation image 41.

The segmentation in the segmentation stage 40 can be efficiently performed by threshold value formation, for example, since the signal, the vascular tree 12, and the noise are more strongly separated. Thus, a binary image can be generated in which e.g. the entry “0” stands for background and “1” for vascular tree. The threshold value should be generated as a given factor of the typical noise in the modified vessel image 39.

In a third image processing stage 42 the modified vessel image 39, the segmentation image 41 and/or the object image 29 can be subtracted or overlaid to form a composite image 43, as will be described in more detail below.

Spatial filter methods (e.g. noise suppression methods, sharpness filters, etc.) can be selectively controlled with the aid of the segmentation image, i.e. the choice of parameters adapted as a function of the binary value. In this case not only can the vascular and non-vascular regions be selected, but the mask image M and the interventional images Sₙ (or Fₙ) can also be handled differently.

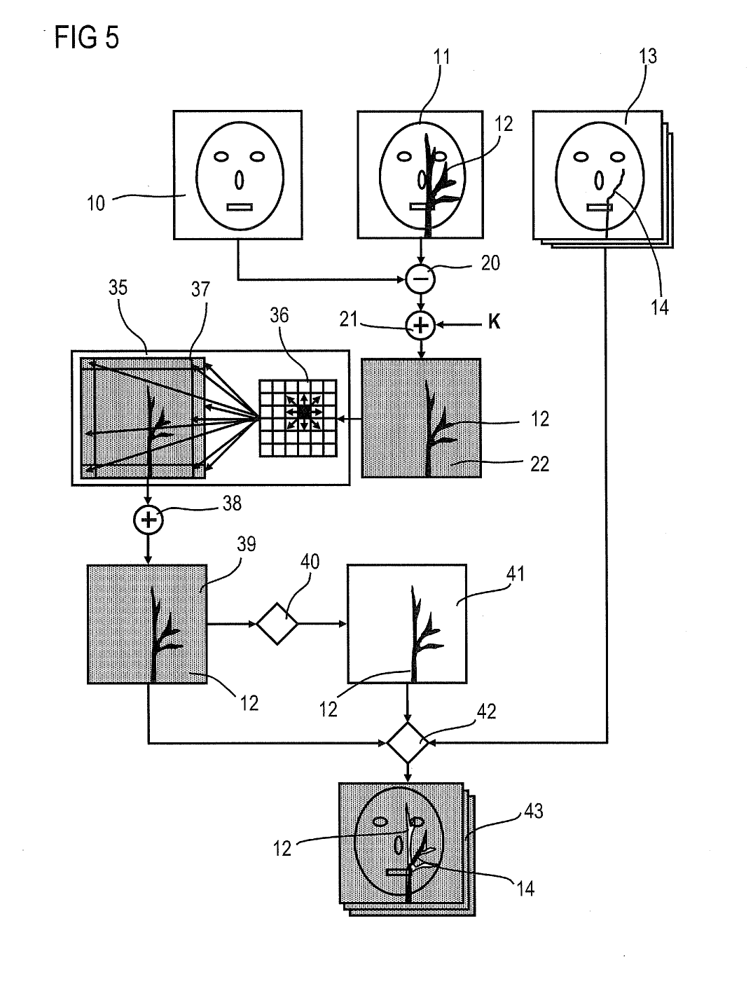

FIG. 5 shows a second embodiment variant of the method according to the invention which is similar to that described with reference to FIG. 4.

The segmentation in the segmentation stage 40 can be performed efficiently by means of threshold value formation, for example, since the signal, the vascular tree 12, and the noise can be separated more strongly. Thus, a binary image can be generated in which e.g. the entry “0” stands for background and “1” for vascular tree. The threshold value should be generated as a given factor of the typical noise in the modified vessel image 39.

In the third image processing stage 42 the modified vessel image 39, the segmentation image 41 and/or the series of fluoroscopic images 13 are overlaid to form an image series of composite images 43, as is yet to be described below.

Instead of a roadmap method a so-called overlay reference method is described in which a mask image M′ and a series of fluoroscopic images Fₙ (13) are overlaid with the aid of the binary information B of the segmentation image 41 in a precisely targeted manner by merging. The overlay reference method is equivalent to other possible overlay method using a mask.

In this case, analogously to the roadmap method, a first subtraction image 22 or mask image M is produced and, by means of the described method, a segmented image B (41) is generated by way of the segmentation stage by displacement (35) and summation (38) of the vessel images 37 displaced relative to one another in a modified vessel image 39 or a new mask M′. The mask image M can originate for example from a subtraction image produced at an earlier time by means of digital subtraction angiography (DSA). The mask image can, however, also come from a 3D DSA or CT acquisition from which a suitable projection has been computed. The mask image can now be overlaid with a series of native images in which for example the interventional phase, in other words, for example, the positioning of the wire, is recorded. The result is not an anatomy-free image, as in the case of a roadmap, but an image which shows more or less anatomy, wire and vascular tree depending on the degree of mixing of mask and native image. This embodiment is referred to as an overlay method or overlay reference method.

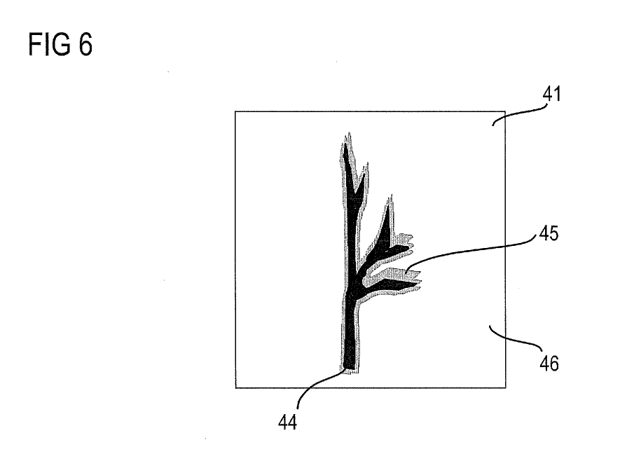

FIG. 6 shows a variant of a segmentation image 41 or binary image B described with reference to FIG. 4, having a softer transition between the vascular and the non-vascular region. In this case the vessels are assigned a weighting of w=2, the transition regions a weighting of w=1, and the remaining region a weighting of w=0.

By means of the method according to the invention described here a self-improving signal-to-noise ratio is obtained for the segmentation of masks in imaging subtraction methods such as, for example, roadmap methods or image overlay methods. The essential aspect of said method is to permit or facilitate a segmentation of the vascular tree for the mask image through “self-amplifying” effects in which signal and noise increase with different factors. The segmentation permits targeted additional image processing steps which can contribute toward improving the image quality of roadmap methods or overlay methods.

The segmentation of the vascular tree is non-trivial, since the signal is relatively small compared to the ambient noise. The potential for segmentation is now made realizable through relative suppression of the noise with respect to the signal. Toward that end the following facts are exploited:
the image noise is essentially uncorrelated from pixel to pixel and
the average pixel value of the non-signals (all points outside of the vascular tree) is described by “zero” or a constant.
Also exploited is the fact that the region of the vascular tree that is generally of interest for interventions has projected thicknesses which are significantly greater than the size of a pixel (e.g. 150 or 200 μm).

A new intermediate image Mij(x+i, y+j) is now generated from an original mask M(x,y)—this can be the mask produced in conventional roadmapping—by displacement of the image by pixels in the x- and/or y-direction by a pixel i and/or j in each case.

The original mask and the intermediate images Mij are then added to form a new mask M′ij and are correctly normalized in the process.

$${M}^{′} ij ( x , y ) = \frac{1}{9} {\sum }_{i , j = - 1}^{i , j = 1} M ( x + i , y + j )$$

More generally, a larger “displacement region” of i=−I, +I and j=−J, +J can be defined, where e.g. I=J=2 or also asymmetrically such as e.g. I=2, J=3 (e.g. in order to take into account the primary orientation of the vascular trees in the corresponding direction). Furthermore, the intermediate images can be weighted differently, e.g. those for which the displacement is spatially greater (e.g. radial distance) having a lower weighting. More generally, this can be expressed as follows:

$${M}^{′} ij ( x , y ) = \frac{1}{{\sum }_{i = - I , j = - J}^{i = I , j = J} {w}_{ij}} {\sum }_{i = - I , j = - J}^{i = I , j = J} {w}_{ij} \cdot M ( x + i , y + j )$$

Since no image information is available in the border zones of the images it is necessary in this case to extrapolate in some way, e.g. by simple continuation (same value as in the last possible pixel). In practice, however, this does not play a significant role since the information of interest (vascular tree) is generally positioned in the center of the image.

The segmentation can now be performed very much more efficiently e.g. through threshold value formation, since signal (vascular tree 12) and noise are separated more strongly. A binary image can therefore be generated in which e.g. the entry “0” stands for background and “1” for vascular tree. The threshold value should be generated as a given factor of the typical noise in the image.

The segmentation can be further improved by searching for related regions and eliminating “islands” formed for example by individual pixels or pixel groups having increased levels of noise.

Other segmentation methods are conceivable.

Generally there is a good spatial overlapping of vessel and wire. However, because the wire has a certain stiffness or if the vessel is characterized by a particularly strong curvature it can happen that the vessel that is filled only with contrast agent and the vessel in which the wire is located do not perfectly overlap. As a result the wire appears “outside” of the vascular tree at said points in the subtraction image.

In order to circumvent this problem the segmentation image 41 or binary image B for example can be modified in such a way that the segmentation region is widened for example by one, two or more pixels. This approach is already achieved in part in any case by the addition method of the mask and its copies displaced by one or more pixels.

Following segmentation of the vascular tree 12 very much more complex and more targeted further processing steps can now be performed than are provided by the classical subtraction of the mask from the series of current X-ray images which show the movement of a wire, for example.

In this case the image processing in the vascular tree and outside of the vascular tree can now be differentiated (this information is contained in the segmentation image), since the wire is located within the vascular tree.

In the following description the new mask is designated by M′, the segmented or binary image by B, and the series of anatomy-corrected fluoroscopic images Fₙ, as subtraction images by Sₙ, wherein the series consists of N images (n=1, N).

More complex image processing steps of this kind can be for example the following:
The subtraction of the new mask M′ from the subtraction image Sₙ is limited to the region of the vascular tree (B(x,y)=1). This avoids the noise level being increased unnecessarily in the remaining regions—these regions in fact carry no information, are therefore “zero” except for noise, since in this case anatomy has been subtracted from anatomy.
The anatomy-corrected fluoroscopic image Sₙ is increased in contrast prior to the subtraction of the mask and moreover in turn only at the point B(x,y)=1. This increase in contrast can be realized by means of a linear or non-linear function. Advantage: The visibility of the wire in the final image Rₙ or composite image 43 is improved. By restricting the increase in contrast to the segmented region the noise in the remaining image is not made worse unnecessarily.
Spatial filter methods (e.g. noise suppression methods, sharpness filters, etc.) can be selectively controlled with the aid of the segmentation image, i.e. the choice of parameters adapted as a function of the binary value. In this case not only can the vascular and non-vascular regions be selected, but the mask image M and the interventional images Sₙ (or Fₙ) can also be handled differently.

Instead of a binary image it is also possible to generate a “segmentation image” which maps a less abrupt transition from vascular tree and remainder region. For example, the vascular tree region could be assigned the value “2”, the neighboring pixels in each case or the neighboring two pixels the value “1”, and the regions then remaining the value “0”, as has been described with reference to FIG. 6. Obviously, other and finer divisions are conceivable. The modification of the parameters of vascular and non-vascular region and hence also the resulting image impression would consequently be less abrupt.

The same method described above for the roadmap method can be employed equivalently for other overlay methods using a mask, for example the so-called overlay reference method, as has been described in connection with FIG. 5. In this case, analogously to the roadmap method, a mask image M is produced and—using the described method—a segmented image B generated. The mask image M can originate e.g. from a digital subtraction angiography (DSA) produced at an earlier time. The mask image can, however, also come from a 3D DSA or CT acquisition from which a suitable projection has been computed. The mask image can now be overlaid with a series of native images in which e.g. the interventional phase, in other words, for example, the positioning of the wire, is recorded. The result is not an anatomy-free image (such as in the case of a roadmap), but an image which shows more or less anatomy, wire and vascular tree depending on the degree of mixing of mask and native image. This embodiment is referred to as an overlay (or overlay reference) method.

In this case, too, complex and more precisely targeted image processing steps can be performed, in similar fashion to those described above, on the basis of the segmented image. For example, the contrast of the mask can be increased or reduced at all points at which the binary image is not equal to “zero”.

This enables the visualization of the interventional region (vascular tree 12 and wire 14, B(x, y)=1) and the surrounding anatomy (B(x,y)=0) to be selectively controlled in a more precisely targeted manner.

1.-12. (canceled)

13. A method for enhancing a visualization of an introduced object in interventional angiographic examinations, comprising:
acquiring an empty image, a fill image having a vascular tree filled with a contrast-agent, and a native image having the object by a detector having a matrix-shaped array of pixels;
subtracting the empty image from the fill image to generate a subtraction image;
displacing the subtraction image by at least one pixel in a x- and/or a y-direction to generate a plurality of vessel images displaced relative to one another;
summing the vessel images to generate a modified vessel image as a mask image having a substantially improved signal-to-noise ratio;
segmenting the vascular tree in the modified vessel image to generate a segmentation image;
processing the modified vessel image, the segmentation image, and the native image to generate a composite image; and
displaying the composite image.

14. The method as claimed in claim 13, further comprising targeted overlaying a fluoroscopic image on the modified vessel image by merging based on binary information of the segmentation image.

15. The method as claimed in claim 13, further comprising:
subtracting the empty image from the native image to generate a further subtraction image;
processing the further subtraction image to generate an object image; and
processing the modified vessel image, the segmentation image, and the object image to generate the composite image.

16. The method as claimed in claim 15, wherein the modified vessel image, the segmentation image, and the native image or the object image is processed in a region of the vascular tree in the segmentation image.

17. The method as claimed in claim 15, wherein a contrast is increased in a region of the vascular tree in the segmentation image when starting processing the native image or the object image.

18. The method as claimed in claim 15, wherein the segmentation image selectively controls filter methods in the processing step.

19. The method as claimed in claim 18, wherein the filter methods comprise spatial filter methods.

20. The method as claimed in claim 19, wherein the spatial filter methods comprise a noise suppression method and/or a sharpness filter method.

21. The method as claimed in claim 18, wherein the filter methods are selected by the segmentation image to treat a vascular region and a non-vascular region separately.

22. The method as claimed in claim 18, wherein the filter methods are selected by the segmentation image for processing the modified vessel image, the native image or the object image differently.

23. An angiographic X-ray system for enhancing a visualization of an introduced object in interventional angiographic examinations, comprising:
an X-ray tube assembly;
an X-ray detector having a matrix-shaped array of pixels for acquiring an empty image, a fill image having a vascular tree filled with a contrast-agent, and a native image having the object;
an imaging processing system configured to:
subtract the empty image from the fill image to generate a subtraction image;
displace the subtraction image by at least one pixel in a x- and/or a y-direction to generate a plurality of vessel images displaced relative to one another;
sum the vessel images to generate a modified vessel image as a mask image having a substantially improved signal-to-noise ratio;
segment the vascular tree in the modified vessel image to generate a segmentation image;
process the modified vessel image, the segmentation image, and the native image to generate a composite image; and
a monitor for displaying the composite image.

24. The angiographic X-ray system as claimed in claim 23, wherein the imaging processing system is further configure to:
subtract the empty image from the native image to generate a further subtraction image;
process the further subtraction image to generate an object image; and
process the modified vessel image, the segmentation image, and the object image to generate the composite image.

25. The angiographic X-ray system as claimed in claim 23, wherein the imaging processing system is further configure to targeted overlay a fluoroscopic image on the modified vessel image by merging based on binary information of the segmentation image.

26. The angiographic X-ray system as claimed in claim 23, wherein the angiographic X-ray system is a C-arm X-ray system.
